(19) United States
(12) Patent Application Publication
(10) Pub. No.: US 2026/0265678 A1
(43) Pub. Date: Sep. 10, 2026

(54) AEROTOLERANT BACTERIA FERMENTATION

(71) Applicant: Vaxcyte, Inc., San Carlos, CA (US)

(72) Inventors: Peter T. Davey, Foster City, CA (US); Namita Bansal, Union City, CA (US); Katherine A. Peffen, San Francisco, CA (US); Johanna Leslie V. King, Belmont, CA (US)

(21) Appl. No.: 19/166,324

(22) PCT Filed: Mar. 15, 2024
(86) PCT No.: PCT/US2024/020100

§ 371 Date: Sep. 17, 2025

Related U.S. Application Data
(60) Provisional application No. 63/452,877, filed on Mar. 17, 2023.

(51) Int. Cl. C12N 1/20; C12P 19/04; C12R 1/46

(52) U.S. Cl. CPC C12N 1/20; C12P 19/04; C12N 2500/02; C12R 2001/46

(57) ABSTRACT

Described herein are processes for the fermentation of aerotolerant anaerobic bacteria. Such processes may result improved production and isolation of biomolecules. Isolated biomolecules may include capsular polysaccharides for use in the production of immunogenic compositions.

### CROSS REFERENCE TO RELATED APPLICATIONS

This application claims priority to U.S. Patent Application No. 63/452,877, filed on Mar. 17, 2023, the contents of which are incorporated herein in their entirety.

### TECHNICAL FIELD

This invention relates generally to the fermentation of aerotolerant anaerobes. The invention relates to improving fermentation conditions for aerotolerant anaerobes such as Streptococcus pyogenes, Streptococcus agalactiae, Streptococcus pneumoniae, Staphylococcus, Enterococcus, Clostridia, and Shigella. In particular, the invention relates to novel improved fermentation conditions for producing capsular polysaccharides from aerotolerant anaerobes, where the capsular polysaccharides may further be used in immunogenic compositions for preventing and/or treating infection caused by the bacteria.

### BACKGROUND

### Aerotolerant Anaerobic Bacteria

Aerotolerant anaerobes are bacteria that do not require oxygen for growth and can tolerate some amount of oxygen during the growth stage. Aerotolerant bacteria meet their energy demands through fermentation, meaning they use fermentation to make ATP for energy.

Aerotolerant bacteria are able to survive in the presence of oxygen through evolutionary strategies for protecting themselves. One such way is that they are able to produce enzymes that counter the destructive effects of some forms of oxygen. For example, superoxide dismutase is an enzyme capable of converting a superoxide anion into ground-state oxygen and hydrogen peroxide, where then the hydrogen peroxide is degraded by a second enzyme, e.g., catalase or peroxidase. Examples of aerotolerant anaerobes include Streptococci and Lactobacillus species.

Streptococcus pneumoniae

One example of the aerotolerant bacteria Streptococci species is Streptococcus pneumoniae. Streptococcus pneumoniae is a species of Gram-positive bacteria. These bacteria are extracellular and made up of non-motile and non-sporing cocci that typically link in chains. Streptococcus pneumoniae is the main cause of community-acquired pneumonia and meningitis in children and the elderly, and sepsis in those infected with HIV. Streptococcus pneumoniae also causes many types of pneumococcal infections other than pneumonia. These invasive pneumococcal diseases include bronchitis, rhinitis, acute sinusitis, otitis media, conjunctivitis, meningitis, sepsis, osteomyelitis, septic arthritis, endocarditis, peritonitis, pericarditis, cellulitis, and brain abscess. FIG. 1 shows Streptococcus pneumoniae under magnified conditions.

Streptococcus pyogenes

Streptococcus pyogenes (GAS) is another example of an aerotolerant bacteria. GAS, while infrequent in humans, is typically pathogenic and causes Group A streptococcal infections. An estimated 700 million GAS infections occur worldwide every year. GAS increases cases of severe invasive infections, sepsis, necrotizing fasciitis, otitis media, and toxic shock syndrome. Pharyngitis is highly prevalent in school-aged children and a major source of antibiotic prescriptions worldwide, which drives selective pressure for resistance throughout the human microflora. GAS is also responsible for post-infectious immune-mediated rheumatic heart disease (RHD), a leading cause of mortality in the developing world. Some 30 million people are currently affected by RHD, with over 300,000 deaths annually (60%<age 70) and 11.5 million disability-adjusted life years lost.

Currently, there is still much research and development around effective vaccines for Streptococcus pneumoniae and there are no vaccines on the market that are directed specifically toward GAS. At least for Streptococcus pneumoniae, effective vaccine compositions require making different Streptococcus pneumoniae serotypes through fermentation. Fermentation is a primary step in the production of the various serotypes used in creating vaccine conjugates on the market and has a major impact on the ability to make enough of each serotype for the vaccine compositions. While the media used in fermentation plays an important role in the process, certain serotypes, regardless of the type of media used still fail to grow to an acceptable concentration for effective scale-up and manufacturing. Thus, there is still a need to develop ideal methods for fermenting certain hard-to-grow serotypes of various aerotolerant bacteria(s).

### SUMMARY

Described herein are methods for culturing aerotolerant bacteria for making a biomolecule (e.g., cell wall polysaccharide). The process can include the following steps of: inoculating a culture medium with a colony or preculture of the aerotolerant bacteria in a bioreactor to form a bacterial culture, supplying the bioreactor with a gaseous mixture substantially free of oxygen, wherein the gaseous mixture comprises at least one of nitrogen (N₂) and carbon dioxide (CO₂), incubating the bacterial culture, and maintaining a relatively oxygen-free environment in the bioreactor. In some instances, the gas mixture employed during the fermentation process is substantially free of any oxygen. This means that the container or bioreactor where the fermentation occurs will be substantially free of any oxygen. In some instances, the chamber or bioreactor used for fermentation is first purged with a gaseous mixture substantially free of oxygen. The gaseous mixture may be supplied through a pipe assembly or by a sparging mechanism. The system may be purged for 0.1 vvm overnight. The oxygen-free environment can be maintained for the entire duration of the fermentation process or it may be maintained for a period until a threshold amount of the desired product is obtained (e.g., OD₆₀₀ is a certain threshold value). In some instances, the OD₆₀₀ is above 6. In certain instances, a gaseous mixture that is substantially free of oxygen is supplied directly to the culture medium or bacterial culture contained within the bioreactor. In some instances, the gaseous mixture is 90-95% N₂ and about 5-10% CO₂. In other instances, the gaseous mixture is 95% N₂ and 5% CO₂. In some instances, the repeatability of the anaerobe bacteria growth profiles is improved with an environment of 90-95% N₂ and about 5-10% CO₂ as compared to an environment with 5% CO₂ and 95% ambient air.

For culturing the aerotolerant bacteria, a biological temperature range of between 30° C. and 39° C. is maintained. In some instances, a temperature of 37° C. is maintained throughout the entire process.

In some examples, the culture process occurs in a culture medium (e.g., agar medium). In some examples, the culture medium is substantially free of sugar, vegetable oil, hydrocarbon, alcohol, or organic acid added to the culture medium and/or bacterial culture.

In some examples, the bacteria culture includes at least one nitrogen source. In some examples, the bacterial culture includes at least one nitrogen source selected from urea, ammonium hydroxide, ammonium salts, nitrates, amino acids, yeast extract, yeast autolysates, yeast nitrogen bases, protein hydrolysates, soybean meal, Hy-Soy, tryptic soy broth, cotton seed meal, malt extract, corn steep liquor, and molasses.

In some examples, the bacterial culture comprises at least one phosphorus source. In some examples, the bacterial culture includes at least one phosphorus source where the phosphorus source is a phosphate or polyphosphate.

In some examples of the aerotolerant bacteria culturing process, the bacterial culture does not comprise an animal-derived material. Examples of animal-derived materials may include animal-derived heme or a prion protein.

The above-mentioned process may be used for aerotolerant bacteria such as S. pneumoniae or S. pyogenes. Specifically, for S. pneumoniae, serotypes may be selected from 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20, 20A, 20B, 21, 22F, 23A, 23B, 23F, 24F, 31, 33F, 34, 35B, 35F, and 38. For S. pyogenes, serotypes may be selected from M1, M2, M3, M4, M5, M6, M9, M11, M12, M13, M18, M22, M25, M28, M62, M71, M72, M74, M75, M77, M80, M81, M83, M87, M89, and M92. For S. pyogenes, the biomolecule produced may be lacking an immunodominant N-acetyl Glucosamine (GLcNAc) side chain.

The biomolecule produced using the fermentation conditions described may be isolated and/or purified for use in a pharmaceutical composition that may be administered to a subject.

### BRIEF DESCRIPTION OF THE DRAWINGS

FIG. 1 shows a picture of Streptococcus pneumoniae under magnification.

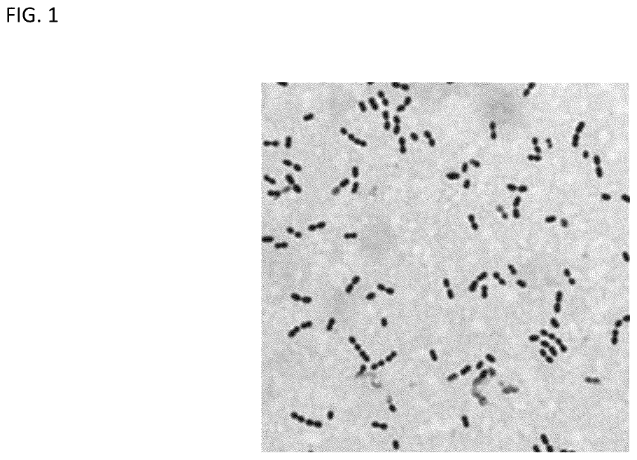

FIG. 2 shows a graphical representation of an overall fermentation process for growing various Streptococcus pneumoniae serotypes.

FIG. 3 is a plot of Streptococcus pneumoniae serotype 15A growth curves under various conditions as described in Example 2.

FIG. 4A is a plot of Streptococcus pneumoniae serotype 23A growth under various fermentation conditions as described in Example 2.

FIG. 4B is a series of plots for Streptococcus pneumoniae serotype 23A showing dissolved oxygen against time where the fermentation processes were run at conditions with 5% CO2 and 95% ambient air as described in Example 2.

FIG. 4C shows a series of plots of OD₆₀₀ over time at different flow rates of 95% N₂ and 5% CO₂ for Streptococcus pneumoniae serotype 23A, as described in Example 2.

FIG. 4D shows a series of plots of Streptococcus pneumoniae serotype 23A capsular polysaccharide production over time at four different flow rates of 95% N₂ and 5% CO₂, as described in Example 2.

FIG. 5 is a plot of Streptococcus pneumoniae serotype 18C growth under various fermentation conditions as described in Example 2.

### DETAILED DESCRIPTION

As used herein, an anaerobe is a bacteria that does not require oxygen for growth. Anaerobes may be obligate anaerobes, which could possibly react negatively to, or even die, in the presence of oxygen. The other types are the facultative anaerobes that make ATP by aerobic respiration if oxygen is present else grow in its absence also. Streptococcus pneumoniae is a facultative anaerobe, meaning that it can grow in either the presence or absence of oxygen.

As used herein, “batch fermentation” is one of the methods used in the industrial production of microorganisms, where a sterile culture medium is inoculated with the microorganisms and no additional growth medium is added.

As used herein, “culture medium” is a liquid or gel designed to support the growth of the microorganisms or cells. Such a medium may be customized to meet the specific requirements of growth of the bacteria.

In certain embodiments, the present invention is directed to the growth and production of polysaccharides from S. pneumoniae. Preferably the serotypes selected are one or more, or all, of 1, 2, 3, 4, 5, 6A, 6B, 7C, 7F, 8, 9N, 9V, 10A, 11A, 12F, 14, 15A, 15B, 16F, 17F, 18C, 19A, 19F, 19A, 20A, 20B, 22F, 23A, 23B, 23F, 31, 33F, and 35B. The strain used may be a wild-type or genetically modified strain.

In other embodiments, the described fermentation conditions can be applied to the growth and production of polysaccharides from the Group A Streptococcus bacteria is of a serotype selected from M1, M2, M3, M4, M6, Mi 1, M12, M22, M28, M75, and M89. In some embodiments, the GAS bacterium is a serotype selected from M1, M3, M5, M9, M12, M18, M22, M25, M28, M71, M72, and M74. In some embodiments, the GAS bacterium is a serotype selected from M1, M4, M6, Mi 1, M12, M22, M44, M75, M77, M77, and M81. In some embodiments, the GAS bacterium is a serotype selected from M1, M2, M3, M4, M6, M9, M12, M18, M22, M75, M77, M89, and M92. In some embodiments, the GAS bacterium is a serotype selected from M1, M2, M3, M4, M5, M6, M9, Mi 1, M12, M13, M28, M62, and M89. In some embodiments, the GAS bacterium is a serotype selected from M1, M2, M3, M4, M6, M12, M22, M28, M49, M53, M68, M77, M80, M83, M87, M89, and M92.

As used herein, “fed-batch fermentation” is a method for the industrial production of microorganisms wherein, the growth-limiting substrate is continuously added to the fermentation vessel. This growth-limiting substrate can be anything including carbon or nitrogen source, vitamins, essential amino acids, etc.

Fed-batch can be a fixed volume or a variable volume fed-batch. In the fixed volume fed-batch the volume of the fermentation medium is kept almost the same by either supplying the feed in an undiluted form or in form of a gas. Another method of keeping the volume the same is by supplying the feed by dialysis. Cyclic fed-batch culture for fixed-volume systems refers to a periodic withdrawal of a portion of the culture and the use of the residual culture as the starting point for a further fed-batch process. In other words, once the fermentation reaches a certain stage, the culture is removed, and the biomass is diluted to the original volume with sterile water or a medium containing the feed substrate. The dilution decreases the biomass concentration and results in an increase in the specific growth rate.

As used herein, variable volume fed-batch is a fed batch process in which the volume changes with the fermentation time due to the substrate feed. The feed is provided in the same batch fermentation vessel so as to increase the initial volume of the medium. The feed may be the growth-limiting substrate fed at a concentration equal to its concentration in the initial medium or in concentrated form.

As used herein, an “immunogenic composition” is a formulation capable of provoking an immune response when administered to a subject. In some embodiments, the subject is a mammal (e.g., a human).

Pneumococcal conjugate vaccines (PCVs) are effective against Streptococcus pneumoniae. PCVs are designed to cover some of the more prevalent serotypes. In some examples of PCVs, capsular polysaccharides of Streptococcus pneumoniae serotypes are selected from a group consisting of 1, 2, 3, 4, 5, 6A, 6B, 7F, 8, 9N, 9V, 10A, 11A, 12F, 14, 15B, 17F, 18C, 19A, 19F, 20B, 22F, 23F, and 33F. In some other examples of PCVs, capsular polysaccharides of Streptococcus pneumoniae serotypes are selected from a group consisting of 1, 2, 3, 4, 5, 6A, 6B, 7C, 7F, 8, 9N, 9V, 10A, 11A, 12F, 14, 15A, 15B, 16F, 17F, 18C, 19A, 19F, 20B, 22F, 23A, 23B, 23F, 31, 33F, and 35B. PCVs are complex compositions that require the production of each of the serotypes contained within the vaccine composition. Difficulties with or failure to produce one of the desired serotypes can delay production of the PCV formulation. See, for example: International publications WO2018/126229 and WO2020/009993, and US publication US2020/0054739.

Similarly, for Streptococcus pyogenes, development of various vaccines may require the production of various serotypes of S. pyogenes. Various serotypes of S. pyogenes can be selected from M1, M2, M3, M4, M5, M6, M9, M11, M12, M13, M18, M22, M25, M28, M62, M71, M72, M74, M75, M77, M80, M81, M83, M87, M89, and M92. The strain used may be a wild-type or genetically modified strain. For S. pyogenes, certain genetically modified strains may produce a biomolecule lacking an immunodominant N-acetyl Glucosamine (GLcNAc) side chain (see e.g., U.S. Pat. No. 10,780,155 and International publications WO2022/178015 and WO2021/167996).

Described herein are fermentation methods that improve the growth of various aerotolerant bacterial serotypes. The growing conditions for culturing aerotolerant bacteria (such as S. pneumoniae) are paramount to the production of large amounts of the different serotypes, especially for vaccine production. Currently accepted fermentation methods utilize a mixture of air and CO₂. Inventors here found that an environment essentially free (e.g., less than about 2%, less than about 1%, or 0% oxygen) of any oxygen resulted in better growth of certain serotypes of S. pneumoniae. This was unexpected because S. Pneumoniae typically lives in the mucus layer overlying the epithelial surface of the upper respiratory tract in humans/mammals (e.g., the nose and throat regions) where there is always the presence of oxygen. Thus, it was surprising that serotypes of S. pneumoniae grew significantly better in conditions essentially free of oxygen. Described herein are fermentation methods for enhanced growth outcomes of various aerotolerant bacteria including but not limited to Streptococci species such as S. pneumoniae and S. pyogenes, and Lactobacillus species.

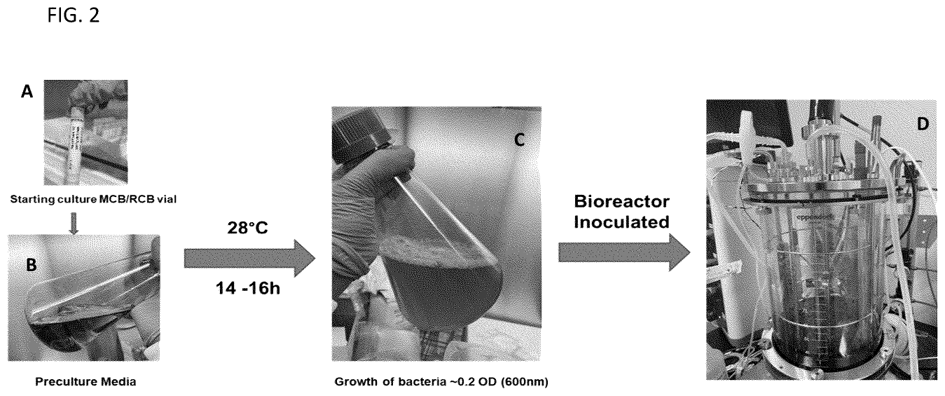

FIG. 2 shows pictures of an exemplary fermentation process. Fermentation for each individual serotype begins with a starting culture supplied through various external sources (starting serotype cultures were obtained from master cell bank/research cell bank MCB/RCB) from Lonza Group AG (FIG. 2A). The starting culture (~2 μL) is then introduced into the pre-culture media (FIG. 2B) and allowed to grow at 28° C. for between 14 to 16 hours, or until the OD₆₀₀ (via spectrophotometer) of the solution is 0.2 (see FIG. 2C). The pre-culture solution may be maintained at temperatures between 28° C. and 39° C. The pre-culture solution may be shaken at 120-140 RPM. The resulting solution is then introduced into a bioreactor that holds batch medium (FIG. 2D) where the batch medium solution may be purged or sparged to remove oxygen. Under some conditions described herein, for various serotypes, reactors containing batch medium can be inoculated with a particular pre-culture. Table 1 below is an exemplary embodiment of a pre-culture medium composition.

TABLE 1
Pre-culture Medium (BML Density
1.0107) Final pH 7.6 Flask media

| | Final | | |
|---|---|---|---|
| | Concentration | | Component acts as a |
| Compound | (g/L) | | source for: |
| Bacto Yeast Extract | 20 | | Amino acid (AA), vitamin B, |
| Ultrafiltrate | | | some trace elements, carbon |
| | | | and nitrogen |
| Hy-Soy peptone | 10 | | Nitrogen |
| Salt solution (Ca/Mg) | 20 | mL | Metals |
| KH₂PO₄ | 0.02 | | Phosphates, buffering |
| K₂HPO₄ | 0.02 | | Phosphates, buffering |
| NaHCO₃ | 0.6 | | Carbonate ions that are |
| | | | essential for the metabolic |
| | | | functions of most cells |
| NaCl | 0.04 | | Salt |
| Glucose Alternative | 9.1 | | Carbon |
| (Dextrose anhydrous) | | | |
| L-Cysteine-HCl•H₂O | 11.43 | mL | Amino acid |

| (175 g/L stock) | | |
|---|---|---|
| HEPES | 47.7 | Buffer system |

In some embodiments, a batch medium can be inoculated with one pre-culture. Table 2 below is an exemplary embodiment of a batch medium composition:

TABLE 2
Batch Medium (BML density: 1.016,
Final pH 7.0) Bioreactor media

| | Final | | |
|---|---|---|---|
| | Concentration | | |
| Compound | (g/L) | | Sources |
| Bacto Yeast Extract | 10 | | Amino acid, vitamin B, some |
| Ultrafiltrate | | | trace elements, carbon and |
| | | | nitrogen |
| Hy-Soy peptone | 15 | | Nitrogen |
| Salt solution (Ca/Mg) | 20 | mL | Metals |
| KH2PO4 | 0.02 | | Phosphate |
| K2HPO4 | 0.02 | | Phosphate |
| NaHCO3 | 0.4 | | carbonate ions that are essential |
| | | | for the metabolic functions of |
| | | | most cells |
| NaCl | 0.04 | | Salt |
| Alternative (Dextrose | 9.10 | | Carbon |
| Anhydrous) | | | |
| L-Cysteine-HCl•H2O | 0.85 | | Amino acid |
| Trace element solution | 10 | mL | Trace metals |

| PPG 2000 (2000 | 1 | Antifoam |
|---|---|---|
| molecular weight | | |
| polypropylene glycol) | | |

In some embodiments, at the end of a batch phase, exponential feeds of glucose (Feed 1) and complex components (Feed 2) may occur over a 4 h period. Here exponential feeds are based upon the feeding rates are given by Equations 1 and 2, where F_{0,1} and F_{0,2} represent the respective feed rates at feed start, and μ_{SET} the rate of exponential increase over time. Note that the two feed solutions (Feed 1 and Feed 2) are added in parallel with the same starting time.

$${F}_{1} ( t ) = {F}_{0 , 1} {e}^{{μ}_{s e t} * t} \qquad Equation 1$$

$${F}_{2} ( t ) = {F}_{0 , 2} {e}^{{μ}_{s e t} * t} \qquad Equation 2$$

The following Table 3 provides information about the composition of Feed 1 and Feed 2:

TABLE 3

| | Conc (g/L) | Source |
|---|---|---|
| Feed 1 | | |
| MgSO4 (g/L) | 1.07 | Provides ions helpful for |
| | | growth of bacteria |
| D(+) Glucose | 363.6 | Carbon |
| Feed 2 | | |
| Hy soy | 150 | Nitrogen |
| Yeast BD | 30 | AA, carbon, nitrogen |
| Cysteine | 2 | AA |

| Base | Concentration(g/L) | Source |
|---|---|---|
| Na2CO3, 20% (w/v) | 200 | Base and carbon source |

In some embodiments of the present fermentation methods, the growth environment within the bioreactor is essentially free of oxygen. Prior to a fermentation process, the bioreactor is purged, and the media is sparged with N2 and CO2 gas for 12-14 hours or overnight. An O2 sensor may be used to monitor the amount of O2 within the bioreactor. In some instances, the amount of oxygen present in the bioreactors is measured by the volumetric mass transfer coefficient (kLa). The kLa value may be calculated from the amount of dissolved oxygen (DO) as sensed by the O2 sensor. kLa indicates the rate of oxygen used for fermentation where all oxygen-consuming variables within the bioreactor are taken into account. The kLa value provides a measure of oxygen rate transfer within a bioreactor taking into account the presence of media, cells, product formation, ionic species, antifoaming agents, and so forth. kLa can be represented by the following equation:

$$\frac{dCL}{dt} = kLa ( C * - CL )$$

Where CL is the dissolved oxygen concentration and C* is the saturated dissolved oxygen concentration in solution. It should be noted that the kLa value depends upon gas flow rate, the amount of mixing/agitation, and the process for introducing components to the fermentation mixture in the bioreactor. In the case of S. pneumoniae, the kLa has a narrow range for growth that will depend on such factors such as gas flow rate, agitation, and introduction process of additional components used in the fermentation process. Because S. pneumoniae typically resides in an oxygen present environment, it was surprising that certain S. pneumoniae serotypes showed improved growth in an oxygen-free environment.

In some embodiments, the conditions in the bioreactor maintain a kLa value no more than 2.63/hr. In some embodiments, a kLa value of less than 2.50/hr is maintained. In some embodiments, a kLa value of less than 2.25/hr is maintained. In some embodiments, a kLa value of less than 2.50/hr is maintained. In some embodiments, a kLa value of less than 2.00/hr is maintained. In other embodiments, a kLa value of 1.80/hr or less is maintained. In other embodiments, a kLa value of 1.60/hr or less is maintained.

In some embodiments of the fermentation conditions for growing aerotolerant bacteria includes maintaining an environment an environment containing nitrogen and carbon dioxide, and essentially free of oxygen. In some embodiments, the bioreactor environment is 90% N₂ and 10% CO₂. In some embodiments, the bioreactor environment is 91% N₂ and 9% CO₂. In some embodiments, the bioreactor environment is 92% N₂ and 8% CO₂. In some embodiments, the bioreactor environment is 93% N₂ and 7% CO₂. In some embodiments, the bioreactor environment is 94% N₂ and 6% CO₂. In some embodiments, the bioreactor environment is 95% N₂ and 5% CO₂. In some embodiments, the bioreactor environment is 96% N₂ and 4% CO₂. In some embodiments, the bioreactor environment is 97% N₂ and 3% CO₂. In some embodiments, the bioreactor environment is 98% N₂ and 2% CO₂. In some embodiments, the bioreactor environment with the percent N₂ and the percent CO₂ is kept constant throughout the fermentation process.

In some embodiments of the present fermentation methods, the temperature within the bioreactor is maintained at biological ranges (e.g., 35° C.-39° C.). In some embodiments of the present fermentation methods, the temperature within the reactor is maintained at the same temperature throughout the fermentation process. In some embodiments of the present fermentation methods, the temperature within the reactor is 35° C. In some embodiments of the present fermentation methods, the temperature within the reactor is 36° C. In some embodiments of the present fermentation methods, the temperature within the reactor is 37° C. In some embodiments of the present fermentation methods, the temperature within the reactor is 38° C. In some embodiments of the present fermentation methods, the temperature within the reactor is 39° C.

In general, for the fermentation processes of this present disclosure, a starting culture from a master cell bank (MCB)/research cell bank (RCB) inoculum is prepared/obtained for a particular serotype. The starting culture is introduced into a preculture media. In some instances, the pre-culture media is introduced into a larger culture media and maintained at a steady temperature (e.g., 28° C.) for 14 hours to 16 hours or until the bacteria reaches an OD₆₀₀ of 0.2. The resulting media is then batch-fed for two rounds. In some instances of the presently disclosed methods, the first feed includes a carbon source and MgSO₄. In some embodiments, the first batch feed includes glucose and MgSO₄.

### Exemplary Pre-Culture Media

In some embodiments of the present fermentation methods, the pre-culture media used includes but is not limited to yeast extract, soy peptone, salt solutions that include calcium or magnesium, NaHCO3, KH2PO4 or K2HPO4, other salts such as NaCl, a glucose alternative such as dextrose, a nitrogen source from L-cysteine HCl·H2O, and buffering solution/agents.

The pre-culture media of the present disclosure do not comprise animal-derived materials and are thus “animal component-free media” or “animal-free media”. These terms are used interchangeably herein and refer to a culture medium that is devoid of any animal-derived materials. The term “culture medium” refers to a liquid or gel (e.g., agar) designed to support the

Growth of microorganisms or cells. Such a medium may be customized to meet specific requirements of growth of the bacteria and/or the purpose of its growth. The term is inclusive of “agar medium”, which refers to a solid or semi-solid culture medium such as the agar medium used during the initial plating phases of bacterial cultivation, and “liquid culture medium” such as the liquid medium used in the later growth and fermentation phases of bacterial cultivation. The terms “liquid medium” and “liquid culture medium” are also used interchangeably throughout the present disclosure. In some embodiments, the culture media of the present disclosure include agar media and liquid animal-free media.

In some embodiments, the culture media of the present disclosure comprise one or more carbon sources selected from, for example, glucose, fructose, lactose, sucrose, dextrose/dextrose (anhydrous), maltodextrins, starch, glycerol, vegetable oils such as soybean oil, hydrocarbons, alcohols such as methanol and ethanol, and organic acids such as acetic acid. In some embodiments, the carbon source is selected from glucose, glycerol, lactose, fructose, sucrose, and soybean oil. The term “glucose” includes glucose syrups, e.g., glucose compositions comprising glucose oligomers. The carbon source may be added to the culture as a solid or liquid. The amounts carbon sources added to the culture media are those such as known by the skilled artisan and/or present in commercially available media.

In some embodiments, the pre-culture media of the present disclosure includes a carbonate source that is essential for the metabolic functions of most cells. In some embodiments the carbonate source is NaHCO₃. In some embodiments, the concentration of NaHCO₃ is between 0.4 g/L and 0.8 g/L. In some embodiments, the concentration of NaHCO₃ is between 0.5 g/L and 0.7 g/L. In some embodiments, the concentration of NaHCO₃ is 0.5 g/L, 0.6 g/L, 0.7 g/L, or 0.8 g/L. In some embodiments, the concentration of NaHCO₃ is 0.5 g/L. In some embodiments, the concentration of NaHCO₃ is 0.6 g/L. In some embodiments, the concentration of NaHCO₃ is 0.7 g/L. In some embodiments, the concentration of NaHCO₃ is 0.8 g/L.

In some embodiments, the carbon source is a glucose alternative (such as dextrose) in the pre-culture media. In some embodiments, the carbon source is present at a concentration of at least about 5.0 g/L. In some embodiments, the carbon source in the pre-culture media is present at a concentration of between about 5.0 g/L and about 20.0 g/L, about 5.0 g/L and about 15.0 g/L, about 5.0 g/L and about 10.0 g/L, about 10 g/L and about 20 g/L, about 15 g/L and about 20 g/L, or about 10 g/L and about 15 g/L. In some embodiments, the carbon source in the pre-culture media is present at a concentration of about 5.0 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, or 20 g/L. In some embodiments, the carbon source in the pre-culture media is between 7 g/L and 12 g/L. In some embodiments, the carbon source in the pre-culture media is between 8.0 g/L and 10.0 g/L. In some embodiments, the carbon source in the pre-culture media is between approximately 8.5 g/L and 9.5 g/L. In some embodiments, the carbon source in the pre-culture media is 8.6 g/L. In some embodiments, the carbon source in the pre-culture media is 8.7 g/L. In some embodiments, the carbon source in the pre-culture media is 8.8 g/L. In some embodiments, the carbon source in the pre-culture media is 8.9 g/L. In some embodiments, the carbon source in the pre-culture media is 9.0 g/L. In some embodiments, the carbon source in the pre-culture media is 9.1 g/L. In some embodiments, the carbon source in the pre-culture media is 9.2 g/L. In some embodiments, the carbon source in the pre-culture media is 9.3 g/L. In some embodiments, the carbon source in the pre-culture media is 9.4 g/L. In some embodiments, the carbon source in the pre-culture media is 9.5 g/L.

### Nitrogen Source

In some embodiments, the pre-culture media of the present disclosure comprise one or more nitrogen sources selected from, for example, urea, ammonium hydroxide, ammonium salts (such as ammonium sulphate, ammonium phosphate, ammonium chloride, and ammonium nitrate), other nitrates, amino acids such as glutamate and lysine, yeast extract, yeast autolysates, yeast nitrogen base, protein hydrolysates (including, but not limited to peptones, casein hydrolysates such as tryptone and casamino acids), soybean meal, Hy-Soy, tryptic soy broth, cotton seed meal, malt extract, corn steep liquor, and molasses. The amounts of nitrogen sources added to the culture media are those such as known by the skilled artisan and/or present in commercially available media.

In some embodiments, the pre-culture media also includes yeast extract (e.g., Bacto Yeast Extract Ultrafiltrate). The yeast extract is a source of amino acids, vitamin B, carbon, nitrogen, and other trace elements. In some embodiments, the yeast extract is a bacto yeast extract. In some embodiments, the yeast extract is present at a concentration of at least about 5 g/L. In some embodiments, the yeast extract is present at a concentration of about 5 g/L to about 25 g/L, about 5 g/L to about 20 g/L, about 5 g/L to about 15 g/L, about 5 g/L to about 10 g/L, about 10 g/L to about 25 g/L, about 10 g/L to about 20 g/L, or about 10 g/L to about 15 g/L. In some embodiments, the yeast extract is present at a concentration of about 5 g/L, about 10 g/L, about 15 g/L, about 20 g/L, or about 25 g/L.

In some embodiments, the nitrogen source in the pre-culture media is soy peptone (e.g Hy-Soy peptone). In some embodiments, the soy peptone is present at a concentration of at least about 5 g/L. In some embodiments, the soy peptone is present at a concentration of about 5 g/L to about 25 g/L, about 5 g/L to about 20 g/L, about 5 g/L to about 15 g/L, about 5 g/L to about 10 g/L, about 10 g/L to about 25 g/L, about 10 g/L to about 20 g/L, or about 10 g/L to about 15 g/L. In some embodiments, the soy peptone is present at a concentration of about 5 g/L, about 10 g/L, about 15 g/L, about 20 g/L, or about 25 g/L. In some embodiments, the soy peptone is Hy-Soy peptone.

In some embodiments, the nitrogen source is an amino acid such as L-cysteine. In some embodiments, the L-cysteine is in the form of L-Cysteine-HCl·H2O. In some embodiments, L-cysteine is present at a final concentration of at least about 0.5 g/L. In some embodiments, L-cysteine is present at a final concentration of about 0.5 g/L to about 5.0 g/L. In some embodiments, L-cysteine is present at a final concentration of about 0.5 g/L to about 5/0 g/L, about 1.0 g/L to about 5.0 g/L, about 2.0 g/L to about 5.0 g/L, about 3.0 g/L to about 5.0 g/L, about 4.0 g/L to about 5.0 g/L, about 1.0 g/L to about 4.0 g/L, about 1.0 g/L to about 3.0 g/L, about 1.0 g/L to about 2.0 g/L, about 2.0 g/L to about 4.0 g/L, about 3.0 g/L to about 4.0 g/L, or about 2.0 g/L to about 3.0 g/L. In some embodiments, L-cysteine is present at a final concentration of about 0.5 g/L, about 1.0 g/L, about 1.5 g/L, about 2.0 g/L, about 2.5 g/L, about 3.0 g/L, about 3.5 g/L, about 4.0 g/L, about 4.5 g/L, or about 5.0 g/L.

### Phosphorus Source

In some embodiments, the pre-culture media of the present disclosure comprise one or more phosphorus sources. The phosphorus may be in the form of a salt, for example, it may be added as a phosphate (such as ammonium phosphate or potassium phosphate) or polyphosphate. In some embodiments, the phosphorus source is potassium phosphate, monobasic (KH₂PO₄). In other embodiments, the phosphorus source is dipotassium phosphate (K₂HPO₄). In some embodiments, the amount of KH₂PO₄ or K₂HPO₄ is between 0.01 g/L and 0.03 g/L. In some embodiments, the amount of KH₂PO₄ or K₂HPO₄ is 0.02 g/L. Other amounts of phosphorus sources added to the culture media may also be appropriate and are known by the skilled artisan and/or present in commercially available media.

### Pre-Culture Media: Other Components

In some embodiments, the pre-culture media can include a salt. In some embodiments, the salt in the culture media is NaCl. In some embodiments of the culture media, the concentration of NaCl is between 0.03 g/L and 0.05 g/L. In some embodiments of the culture media, the concentration of NaCl is between 0.03 g/L. In some embodiments of the culture media, the concentration of NaCl is between 0.04 g/L. In some embodiments of the culture media, the concentration of NaCl is between 0.05 g/L.

In some embodiments, the pre-culture media may also contain additional salts. Non-limiting examples of such salts are CaCl₂, MgSO₄, FeSO₄, ZnSO₄, MgSO₄, K₂HPO₄, KHPO₄. In some instances the concentration of these salts may vary between about 0.4 g/L to about 10 g/L.

In some embodiments, the culture media can include a buffering system. In some embodiments, the buffering system in the culture media can be HEPES. In some embodiments of the culture media, the concentration of HEPES is between 35.5 g/L and 60.0 g/L. In some embodiments of the culture media, the concentration of HEPES is between 40.0 g/L and 60.0 g/L. In some embodiments of the culture media, the concentration of HEPES is between 45.0 g/L and 55.0 g/L. In some embodiments of the culture media, the concentration of HEPES is between 45.0 g/L and 50.0 g/L. In some embodiments of the culture media, the concentration of HEPES is between 46.0 g/L and 48.0 g/L. In some embodiments of the culture media, the concentration of HEPES is between 46.0 g/L, 46.1 g/L, 46.2 g/L, 46.3 g/L, 46.4 g/L, 46.5 g/L, 46.6 g/L, 46.8 g/L, 46.9 g/L, 47.0 g/L, 47.1 g/L, 47.2 g/L, 47.3 g/L, 47.4 g/L, 47.5 g/L, 47.6 g/L, 47.7 g/L, 47.8 g/L, 47.9 g/L, or 48.0 g/L. In some embodiments of the culture media, the concentration of HEPES is 46.0 g/L. In some embodiments of the culture media, the concentration of HEPES is 46.1 g/L. In some embodiments of the culture media, the concentration of HEPES is 46.2 g/L. In some embodiments of the culture media, the concentration of HEPES is 46.3 g/L. In some embodiments of the culture media, the concentration of HEPES is 46.4 g/L. In some embodiments of the culture media, the concentration of HEPES is 46.5 g/L. In some embodiments of the culture media, the concentration of HEPES is 46.6 g/L. In some embodiments of the culture media, the concentration of HEPES is 46.7 g/L. In some embodiments of the culture media, the concentration of HEPES is 0.8 g/L. In some embodiments of the culture media, the concentration of HEPES is 46.9 g/L. In some embodiments of the culture media, the concentration of HEPES is 47.0 g/L. In some embodiments of the culture media, the concentration of HEPES is 47.2 g/L. In some embodiments of the culture media, the concentration of HEPES is 47.3 g/L. In some embodiments of the culture media, the concentration of HEPES is 47.4 g/L. In some embodiments of the culture media, the concentration of HEPES is 47.5 g/L. In some embodiments of the culture media, the concentration of HEPES is 47.6 g/L. In some embodiments of the culture media, the concentration of HEPES is 47.7 g/L. In some embodiments of the culture media, the concentration of HEPES is 47.8 g/L. In some embodiments of the culture media, the concentration of HEPES is 47.9 g/L. In some embodiments of the culture media, the concentration of HEPES is 48.0 g/L.

### Batch Medium

In some embodiments of the fermentation processes described herein, the batch medium comprises yeast extract, soy peptone, KH₂PO₄, K₂HPO₄, NaHCO₃, salts, a carbon source, an amino acid source, a trace element solution, and an anti-foaming agent. In some embodiments of the batch medium, the medium density typically remains the same throughout the reaction process.

In some embodiments of the present fermentation processes, the batch medium can comprise a yeast extract (e.g., Bacto Yeast Extract Ultrafiltrate). In some embodiments, the yeast extract concentration is between 7.0 g/L and 13.0 g/L. In some embodiments, the yeast extract is present at a concentration of at least about 7.0 g/L. In some embodiments, the yeast extract is present at a concentration of about 8.0 g/L to about 12.0 g/L or about 9.0 g/L to about 11 g/L. In some embodiments, the yeast extract is present at a concentration of about 9.0 g/L. In some embodiments, the yeast extract is present at a concentration of about 9.2 g/L. In some embodiments, the yeast extract is present at a concentration of about 9.4 g/L. In some embodiments, the yeast extract is present at a concentration of about 9.6 g/L. In some embodiments, the yeast extract is present at a concentration of about 9.8 g/L. In some embodiments, the yeast extract is present at a concentration of about 10.0 g/L. In some embodiments, the yeast extract is present at a concentration of about 10.2 g/L. In some embodiments, the yeast extract is present at a concentration of about 10.4 g/L. In some embodiments, the yeast extract is present at a concentration of about 10.6 g/L. In some embodiments, the yeast extract is present at a concentration of about 10.8 g/L. In some embodiments, the yeast extract is present at a concentration of about 11.0 g/L.

In some embodiments of the present fermentation processes, the batch medium comprises at least one nitrogen source. In some embodiments, examples of possible nitrogen sources is similar to what is disclosed above. In some embodiments of the present fermentation process, the nitrogen source can be soy peptone (e.g., Hy-Soy peptone). In some embodiments of the present fermentation process, the soy peptone (e.g., Hy-Soy peptone) concentration in the batch medium is between 10 g/L and 20 g/L. In some embodiments, the soy peptone is present at a concentration of about 11 g/L to about 19 g/L, about 12 g/L to about 18 g/L, about 13 g/L to about 17 g/L, or about 12 g/L to about 16 g/L. In some embodiments, the soy peptone is present at a concentration of about 10 g/L. In some embodiments, the soy peptone is present at a concentration of about 11 g/L. In some embodiments, the soy peptone is present at a concentration of about 12 g/L. In some embodiments, the soy peptone is present at a concentration of about 13 g/L. In some embodiments, the soy peptone is present at a concentration of about 14 g/L. In some embodiments, the soy peptone is present at a concentration of about 15 g/L. In some embodiments, the soy peptone is present at a concentration of about 16 g/L. In some embodiments, the soy peptone is present at a concentration of about 17 g/L. In some embodiments, the soy peptone is present at a concentration of about 18 g/L. In some embodiments, the soy peptone is present at a concentration of about 19 g/L. In some embodiments, the soy peptone is present at a concentration of about 20 g/L. In some embodiments, the soy peptone is Hy-Soy peptone.

In some embodiments of the present fermentation processes, the batch medium comprises at least one phosphorus source. The phosphorus may be in the form of a salt, for example, it may be added as a phosphate (such as ammonium phosphate or potassium phosphate) or polyphosphate. In some embodiments, the phosphorus source is potassium phosphate, monobasic (KH₂PO₄). In other embodiments, the phosphorus source is dipotassium phosphate (K₂HPO₄). In some embodiments, the amount of KH₂PO₄ or K₂HPO₄ is between 0.01 g/L and 0.03 g/L. In some embodiments, the amount of KH₂PO₄ or K₂HPO₄ is 0.02 g/L. Other amounts of phosphorus sources added to the culture media may also be appropriate and are known by the skilled artisan and/or present in commercially available media.

In some embodiments of the present fermentation process, the batch medium comprises at least one source of carbonate ions. In some embodiments the carbonate source is NaHCO₃. In some embodiments, the concentration of NaHCO₃ is between 0.3 g/L and 0.5 g/L. In some embodiments, the concentration of NaHCO₃ is 0.3 g/L. In some embodiments, the concentration of NaHCO₃ is 0.4 g/L. In some embodiments, the concentration of NaHCO₃ is 0.5 g/L.

In some embodiments of the present fermentation process, the batch medium comprises a certain concentration of salt. In some embodiments, the salt in the culture media is NaCl. In some embodiments of the culture media, the concentration of NaCl is between 0.03 g/L and 0.05 g/L. In some embodiments of the culture media, the concentration of NaCl is between 0.03 g/L. In some embodiments of the culture media, the concentration of NaCl is between 0.04 g/L. In some embodiments of the culture media, the concentration of NaCl is between 0.05 g/L.

In some embodiments of the present fermentation process, the batch medium comprises at least one source of carbon ions. In some embodiments, the carbon source is a glucose alternative (such as dextrose) in the pre-culture media. In some embodiments, the carbon source is present at a concentration of at least about 5.0 g/L. In some embodiments, the carbon source in the pre-culture media is present at a concentration of between about 5.0 g/L and about 20.0 g/L, about 5.0 g/L and about 15.0 g/L, about 5.0 g/L and about 10.0 g/L, about 10 g/L and about 20 g/L, about 15 g/L and about 20 g/L, or about 10 g/L and about 15 g/L. In some embodiments, the carbon source in the pre-culture media is present at a concentration of about 5.0 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, or 20 g/L. In some embodiments, the carbon source in the pre-culture media is between 7 g/L and 12 g/L. In some embodiments, the carbon source in the pre-culture media is between 8.0 g/L and 10.0 g/L. In some embodiments, the carbon source in the pre-culture media is between 8.5 g/L and 9.5 g/L. In some embodiments, the carbon source in the pre-culture media is 8.6 g/L. In some embodiments, the carbon source in the pre-culture media is 8.7 g/L. In some embodiments, the carbon source in the pre-culture media is 8.8 g/L. In some embodiments, the carbon source in the pre-culture media is 8.9 g/L. In some embodiments, the carbon source in the pre-culture media is 9.0 g/L. In some embodiments, the carbon source in the pre-culture media is 9.1 g/L. In some embodiments, the carbon source in the pre-culture media is 9.2 g/L. In some embodiments, the carbon source in the pre-culture media is 9.3 g/L. In some embodiments, the carbon source in the pre-culture media is 9.4 g/L. In some embodiments, the carbon source in the pre-culture media is 9.5 g/L.

In some embodiments of the present fermentation process, the batch medium comprises at least one source of amino acid. In some embodiments, an amino acid source is L-cysteine. In some in the form of L-Cysteine-HCl·H2O. In some embodiments, L-cysteine is present at a concentration of at least about 0.5 g/L. In some embodiments, L-cysteine is present at a concentration of about 0.5 g/L to about 1.0 g/L. In some embodiments, L-cysteine is present at a concentration of about 0.5 g/L to about 0.9 g/L, about 0.6 g/L to about 0.9 g/L, or about 0.7 g/L to about 0.9 g/L. In some embodiments, L-cysteine is present at a concentration of about 0.50 g/L. In some embodiments, L-cysteine is present at a concentration of about 0.55 g/L. In some embodiments, L-cysteine is present at a concentration of about 0.60 g/L. In some embodiments, L-cysteine is present at a concentration of about 0.65 g/L. In some embodiments, L-cysteine is present at a concentration of about 0.70 g/L. In some embodiments, L-cysteine is present at a concentration of about 0.75 g/L. In some embodiments, L-cysteine is present at a concentration of about 0.80 g/L. In some embodiments, L-cysteine is present at a concentration of about 0.85 g/L. In some embodiments, L-cysteine is present at a concentration of about 0.90 g/L.

In some embodiments of the present fermentation process, the batch medium comprises at least one source of trace elements. In some embodiments, the trace elements are provided in solution form. In some embodiments, the trace element solution includes Fe₂SO₄, ZnSO₄, and Mn₂SO₄. In some examples, the concentration of the components of the trace element solution is about 0.25 g/L to about 5 g/L for each component.

In some embodiments of the present fermentation process, the batch medium further includes at least one anti-foaming agent. In some embodiments, the anti-foaming agent is polypropylene glycol.

In some embodiments of the present fermentation process, the culture media is free of sugar, vegetable oil, hydrocarbons (e.g. mixture of hydrocarbons ranging from butane to farnasene), alcohol, or organic acids.

In some embodiments of the present fermentation process, the culture media is free of animal-derived materials.

In some embodiments of the present fermentation process, the pH of the fermentation mixture is kept essentially neutral (e.g., from about pH 6.8 to about pH 7.2). In some embodiments of the fermentation process, the pH is 6.8. In some embodiments of the fermentation process, the pH is 6.9. In some embodiments of the fermentation process, the pH is 7.0. In some embodiments of the fermentation process, the pH is 7.1. In some embodiments of the fermentation process, the pH is 7.2.

In some embodiments of the present fermentation process, the fermentation process is conducted with stirring/agitation. In some embodiments, the fermentation process is conducted with stirring between 120 and 160 RPM.

In some embodiments of the present fermentation process, the fermentation process is performed at temperatures between approximately 28° C. and 39° C. In some embodiments of the fermentation process, the process is performed at 28° C. In some embodiments of the fermentation process, the process is performed at 29° C. In some embodiments of the fermentation process, the process is performed at 30° C. In some embodiments of the fermentation process, the process is performed at 31° C. In some embodiments of the fermentation process, the process is performed at 32° C. In some embodiments of the fermentation process, the process is performed at 33° C. In some embodiments of the fermentation process, the process is performed at 34° C. In some embodiments of the fermentation process, the process is performed at 35° C. In some embodiments of the fermentation process, the process is performed at 36° C. In some embodiments of the fermentation process, the process is performed at 37° C. In some embodiments of the fermentation process, the process is performed at 38° C. In some embodiments of the fermentation process, the process is performed at 39° C.

### Fed-Batch Phase Media Feeds

As mentioned above, specific serotypes of the aerotolerant bacteria are grown in bioreactors under two fed-batch fermentation steps. In some examples, the first fed-batch phase media includes an ion source such as MgSO₄ a carbon source such as D(+) glucose. In some examples, the concentration of the ion source (e.g., MgSO₄) in the Feed 1 fed-batch phase media is between 0.95 g/L and 1.18 g/L. In some examples of the feed 1, fed-batch phase media, the ion source concentration is about 0.95 g/L. In some examples of the feed 1, fed-batch phase media, the ion source concentration is about 0.97 g/L. In some examples of the feed 1, fed-batch phase media, the ion source concentration is about 0.99 g/L. In some examples of the feed 1, fed-batch phase media, the ion source concentration is about 1.00 g/L. In some examples of the feed 1, fed-batch phase media, the ion source concentration is about 1.01 g/L. In some examples of the feed 1, fed-batch phase media, the ion source concentration is about 1.03 g/L. In some examples of the feed 1, fed-batch phase media, the ion source concentration is about 1.05 g/L. In some examples of the feed 1, fed-batch phase media, the ion source concentration is about 1.07 g/L. In some examples of the feed 1, fed-batch phase media, the ion source concentration is about 1.09 g/L. In some examples of the feed 1, fed-batch phase media, the ion source concentration is about 1.11 g/L. In some examples of the feed 1, fed-batch phase media, the ion source concentration is about 1.13 g/L. In some examples of the feed 1, fed-batch phase media, the ion source concentration is about 1.15 g/L. In some examples of the feed 1, fed-batch phase media, the ion source concentration is about 1.17 g/L.

In some examples of feed 1, fed-batch phase media, the carbon source is D (+) glucose, where the D (+) glucose concentration is between about 325 g/L and 400 g/L. In some examples of feed 1, fed-batch phase media, the carbon source is D (+) glucose and its concentration is about 325 g/L. In some examples of feed 1, fed-batch phase media, the carbon source is D (+) glucose and its concentration is about 330 g/L. In some examples of feed 1, fed-batch phase media, the carbon source is D (+) glucose and its concentration is about 335 g/L. In some examples of feed 1, fed-batch phase media, the carbon source is D (+) glucose and its concentration is about 340 g/L. In some examples of feed 1, fed-batch phase media, the carbon source is D (+) glucose and its concentration is about 345 g/L. In some examples of feed 1, fed-batch phase media, the carbon source is D (+) glucose and its concentration is about 350 g/L. In some examples of feed 1, fed-batch phase media, the carbon source is D (+) glucose and its concentration is about 355 g/L. In some examples of feed 1, fed-batch phase media, the carbon source is D (+) glucose and its concentration is about 360 g/L. In some examples of feed 1, fed-batch phase media, the carbon source is D (+) glucose and its concentration is about 365 g/L. In some examples of feed 1, fed-batch phase media, the carbon source is D (+) glucose and its concentration is about 370 g/L. In some examples of feed 1, fed-batch phase media, the carbon source is D (+) glucose and its concentration is about 375 g/L. In some examples of feed 1, fed-batch phase media, the carbon source is D (+) glucose and its concentration is about 380 g/L. In some examples of feed 1, fed-batch phase media, the carbon source is D (+) glucose and its concentration is about 385 g/L. In some examples of feed 1, fed-batch phase media, the carbon source is D (+) glucose and its concentration is about 390 g/L. In some examples of feed 1, fed-batch phase media, the carbon source is D (+) glucose and its concentration is about 395 g/L. In some examples of feed 1, fed-batch phase media, the carbon source is D (+) glucose and its concentration is about 400 g/L.

A second fed-batch phase media can be used in the fermentation process. The second fed-batch can be provided through a different inlet port that is connected to the bioreactor. The second feed fed-batch phase media can include a nitrogen source such as Hy Soy, a carbon and a nitrogen source such as yeast BD, and an amino acid source such as the amino acid cysteine. In some embodiments of a second feed Fed-batch Phase Media, the feed includes 135 g/L to 165 g/L of Hy soy, 25 to 35 g/L yeast BD, and 1.5 to 2.5 g/L of cysteine. In some examples of the second feed Fed-batch Phase Media, the concentration of Hy soy is about 135 g/L. In some examples of the second feed Fed-batch Phase Media, the concentration of Hy soy is about 140 g/L. In some examples of the second feed Fed-batch Phase Media, the concentration of Hy soy is about 145 g/L. In some examples of the second feed Fed-batch Phase Media, the concentration of Hy soy is about 150 g/L. In some examples of the second feed Fed-batch Phase Media, the concentration of Hy soy is about 155 g/L. In some examples of the second feed Fed-batch Phase Media, the concentration of Hy soy is about 160 g/L. In some examples of the second feed Fed-batch Phase Media, the concentration of Hy soy is about 165 g/L. In some examples of the second feed Fed-batch Phase Media, the concentration of yeast BD is about 25 g/L. In some examples of the second feed Fed-batch Phase Media, the concentration of yeast BD is about 30 g/L. In some examples of the second feed Fed-batch Phase Media, the concentration of yeast BD is about 35 g/L. In some examples of the second feed Fed-batch Phase Media, the concentration of an amino acid such as cysteine is about 1.5 g/L. In some examples of the second feed Fed-batch Phase Media, the concentration of an amino acid such as cysteine is about 2.0 g/L. In some examples of the second feed Fed-batch Phase Media, the concentration of an amino acid such as cysteine is about 2.5 g/L.

In some embodiments of the Fed-batch phase media, a base such as Na₂CO₃, 20% (w/v) may be present in concentrations of approximately 180 g/L to approximately 220 g/L. In some embodiments of the Fed-batch fermentation process, as Na₂CO₃, 20% (w/v) may be present in concentrations of approximately 180 g/L. In some embodiments of the Fed-batch fermentation process, as Na₂CO₃, 20% (w/v) may be present in concentrations of approximately 185 g/L. In some embodiments of the Fed-batch fermentation process, as Na₂CO₃, 20% (w/v) may be present in concentrations of approximately 190 g/L. In some embodiments of the Fed-batch fermentation process, as Na₂CO₃, 20% (w/v) may be present in concentrations of approximately 195 g/L. In some embodiments of the Fed-batch fermentation process, as Na₂CO₃, 20% (w/v) may be present in concentrations of approximately 200 g/L. In some embodiments of the Fed-batch fermentation process, as Na₂CO₃, 20% (w/v) may be present in concentrations of approximately 205 g/L. In some embodiments of the Fed-batch fermentation process, as Na₂CO₃, 20% (w/v) may be present in concentrations of approximately 210 g/L. In some embodiments of the Fed-batch fermentation process, as Na₂CO₃, 20% (w/v) may be present in concentrations of approximately 215 g/L. In some embodiments of the Fed-batch fermentation process, as Na₂CO₃, 20% (w/v) may be present in concentrations of approximately 220 g/L. At the end of the two-step fed-batch processes, the OD₆₀₀ value may be measured. Typically an OD₆₀₀ value of greater than 6 is desirable.

As mentioned above, aerotolerant bacteria, like Streptococcus pneumoniae or Streptococcus pyogenes are typically found in oxygen-present environments. For example, Streptococcus pneumoniae is typically found in nasal passages and lung tissue of mammals. Typically, Streptococcus pneumoniae serotypes would be grown in similar environments in the laboratory. Surprisingly, certain Streptococcus pneumoniae serotypes grew significantly better in conditions devoid of oxygen.

In some embodiments of the processes described herein, the biomolecule of interest is isolated. For instance, bacterial cells may be isolated as a pellet from culture using standard techniques known in the art. One more biomolecules may be separated from the solution (e.g., reaction mixture, culture solution) or from an isolated bacterial pellet. See, for example, WO2021108792A1. An isolated biomolecule may be purified, such that the biomolecule is substantially separated from non-biomolecule components present in a bacterial cell. For instance, an isolated biomolecule may contain less than about 15%, about 14%, about 13%, about 12%, about 11%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, or about 0.01% impurities and/or byproducts.

### ENUMERATED EMBODIMENTS

Embodiment I-1. A process for fermenting aerotolerant bacteria:
The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

Embodiment I-1. A process for culturing aerotolerant bacteria for production of a biomolecule, the process comprising:
a) inoculating a culture medium with a colony or preculture of the aerotolerant bacteria in a bioreactor to form a bacterial culture;
b) supplying the bioreactor with a gaseous mixture substantially free of oxygen, wherein the gaseous mixture comprises at least one of nitrogen (N₂) and carbon dioxide (CO₂);
c) incubating the bacterial culture; and
d) maintaining a substantially oxygen-free environment in the bioreactor.

Embodiment I-2. The process of embodiment I-1, wherein the culture medium is an agar medium.

Embodiment I-3. The process of embodiment I-1 or I-2, wherein there is no sugar, vegetable oil, hydrocarbon, alcohol, or organic acid added to the culture medium and/or bacterial culture.

Embodiment I-4. The process of any one of embodiments I-1 to I-3, wherein the bacterial culture comprises at least one nitrogen source.

Embodiment I-5. The process of any one of embodiments I-1 to I-4, wherein the bacterial culture comprises at least one nitrogen source selected from urea, ammonium hydroxide, ammonium salts, nitrates, amino acids, yeast extract, yeast autolysates, yeast nitrogen bases, protein hydrolysates, soybean meal, Hy-Soy, tryptic soy broth, cotton seed meal, malt extract, corn steep liquor, and molasses.

Embodiment I-6. The process of any one of embodiments I-1 to I-5, wherein the bacterial culture comprises at least one phosphorus source.

Embodiment I-7. The process of any one of embodiments I-1 to I-6, wherein the bacterial culture comprises at least one phosphorus source is a phosphate or polyphosphate.

Embodiment I-8. The process of any one of embodiments I-1 to I-7, wherein the bacterial culture does not comprise an animal derived material.

Embodiment I-9. The process of embodiment I-8, wherein the animal-derived material is animal-derived heme or a prion protein.

Embodiment I-10. The process of any one of embodiments I-1 to I-9, wherein the gaseous mixture substantially free of oxygen is supplied continuously until the biomolecule is formed.

Embodiment I-11. The process of any one of embodiments I-1 to I-10, wherein the gaseous mixture substantially free of oxygen is supplied into a headspace of the bioreactor.

Embodiment I-12. The process of any one of embodiments I-1 to I-10, wherein the gaseous mixture substantially free of oxygen is supplied directly into the culture medium and/or bacterial culture.

Embodiment I-13. The process of any one of embodiments I-1 to I-12, further comprising the step of purging the bacterial culture with the gaseous mixture.

Embodiment I-14. The process of embodiment I-13, wherein the culture medium and/or bacterial culture is purged for at least 12 hours.

Embodiment I-15. The process of any one of embodiments I-13 to I-14, wherein the bacterial culture is purged continuously until incubation is complete.

Embodiment I-16. The process of any one of embodiments I-1 to I-15, wherein the gaseous mixture substantially free of oxygen comprises about 95% N₂ and about 5% CO₂.

Embodiment I-17. The process of any one of embodiments I-1 to I-16, wherein the gaseous mixture substantially free of oxygen comprises about 90-95% N₂ and about 5-10% CO₂.

Embodiment I-18. The process of any one of embodiments I-1 to I-17, wherein the gaseous mixture is substantially free of oxygen comprising a kLa value no more than 1.80.

Embodiment I-19. The process of any one of embodiments I-1 to I-18, wherein incubating the bacterial culture comprises maintaining the temperature between about 30° C. and about 39° C.

Embodiment I-20. The process of embodiment I-19, wherein incubating the bacterial culture comprises maintaining the temperature at about 37° C.

Embodiment I-21. The process of any one of embodiments I-1 to I-20, wherein incubation is complete when the bacterial culture reaches an OD₆₀₀ of at least 6.

Embodiment I-22. The process of embodiments I-1 to I-21, wherein maintaining a substantially oxygen-free environment in the bioreactor comprises keeping oxygen concentration at 0 in the bacterial culture.

Embodiment I-23. The process of any one of embodiments I-1 to I-22, wherein the aerotolerant bacteria are aerotolerant anaerobic bacteria.

Embodiment I-24. The process of any one of embodiments I-1 to I-23, wherein the aerotolerant bacteria are S. pneumoniae or S. pyogenes.

Embodiment I-25. The process of embodiment I-24, wherein the aerotolerant bacteria are S. pneumoniae.

Embodiment I-26. The process of embodiment I-24 or I-25, wherein the S. pneumoniae are of a serotype selected from 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20, 20A, 20B, 21, 22F, 23A, 23B, 23F, 24F, 31, 33F, 34, 35B, 35F, and 38.

Embodiment I-27. The process of any one of embodiments I-1 to I-26, wherein the biomolecule is a capsular polysaccharide.

Embodiment I-28. The process of embodiment I-24, wherein the aerotolerant bacteria are S. pyogenes.

Embodiment I-29. The process of embodiment I-24 or I-28, wherein the S. pyogenes is a serotype selected from M1, M2, M3, M4, M5, M6, M9, M11, M12, M13, M18, M22, M25, M28, M62, M71, M72, M74, M75, M77, M80, M81, M83, M87, M89, and M92.

Embodiment I-30. The process of any one of embodiments I-1 to I-29, wherein the biomolecule is a cell wall polysaccharide.

Embodiment I-31. The process of any one of embodiments I-24 or I-28 to I-29, wherein the biomolecule is a polysaccharide or a variant thereof that lacks an immunodominant N-acetyl Glucosamine (GLcNAc) side chain.

Embodiment I-32. The process of any one of embodiments I-1 to I-31, wherein the biomolecule is isolated.

### EXAMPLES

### Example 1: Fermentation Process for Growing Aerotolerant Bacteria Comprises the Following for Streptococcus pneumoniae

### Preculture:

Precultures with different inoculation ratios (0.008 and 0.004% (v/v)) were prepared with,

Respectively, 50 μL or 25 μL of RCB in 600 mL of preculture medium (3 L shake flask without baffles). The precultures were incubated at 28° C. and 140 rpm in an incubator for ca. 14-15 h so that the total preculture target duration until inoculation of the bioreactors was 15.5 h. The shake flasks that were not used to inoculate the bioreactors were further incubated for 4-8 h in order to determine the maximal OD₆₀₀.

### Fermentation:

For each serotype, two reactors containing 8.45 kg of batch medium were inoculated with one preculture (about 0.55 L after sampling) each. The cultivation conditions are described in the tables below.

The end of a batch phase was defined as the time at which 103 g of base was consumed.

Subsequently, exponential feeds of glucose (Feed 1) and complex components (Feed 2) were applied during 4 h. Samples were taken at the end of the batch phase as well as after 2 and 4 h of exponential feeding for measurements of OD600, glucose and lactate in the culture broth. At the end of the run, the desired OD600 is least 6 and preferably 7.

TABLE 4

| Fermentation parameters | Value | Units |
|---|---|---|
| Media starting volume | 85 170 | mL |
| Agitation | 150 | RPM |
| pH | 7.0 | units |
| Gas flow rate | 0.51 | SLPH (standard liter per hour) |
| % CO2 | 5.0 | % |
| % N₂ | 95 | % |
| Temperature | 37 | ° C. |
| Inoculation volume | 5.2 | mL |
| Feed trigger | 1.6 | mL |
| Feed time | 4 | h |
| DOC concentration | 0.15 | % |
| Antifoam added to vessel | 159 | μL |

TABLE 5
Preculture Medium (BML Density-
1.0107) Final pH-7.6- Flask media

| | Final | | |
|---|---|---|---|
| | Concentration | | Component acts as a |
| Compound | (g/L) | | source of: |
| Bacto Yeast Extract | 20 | | Amino acid (AA), vitamin B, |
| Ultrafiltrate | | | some trace elements, carbon |
| | | | and nitrogen |
| Hy-Soy peptone | 10 | | Nitrogen |
| Salt solution (Ca/Mg) | 20 | mL | Metals |
| KH₂PO₄ | 0.02 | | Phosphates, buffering |
| K₂HPO₄ | 0.02 | | Phosphates, buffering |
| NaHCO₃ | 0.6 | | Carbonate ions that are essential |
| | | | for the metabolic functions of |
| | | | most cells |
| NaCl | 0.04 | | Salt |
| Glucose Alternative | 9.1 | | Carbon |
| (Dextrose anhydrous) | | | |
| L-Cysteine-HCl•H2O | 11.43 | mL | Amino acid |
| (175 g/L stock) | | | |

| | | |
|---|---|---|
| HEPES | 47.7 | Buffer system |

TABLE 6
Batch Medium (BML density-1.016,
Final pH 7.0)- Bioreactor media

| | Final | |
|---|---|---|
| | Concentration | |
| Compound | (1 g/L) | Sources |
| Bacto Yeast Extract | 10 | Amino acid, vitamin B, some |
| Ultrafiltrate | | trace elements, carbon |
| | | and nitrogen |
| Hy-Soy peptone | 15 | Nitrogen |
| Salt solution(Ca/Mg) | 20 mL | Metals |
| KH2PO4 | 0.02 | Phosphate |
| K2HPO4 | 0.02 | Phosphate |
| NaHCO3 | 0.4 | carbonate ions that are essential |
| | | for the metabolic functions of |
| | | most cells |
| NaCl | 0.04 | Salt |
| Alternative (Dextrose | 9.10 | Carbon |
| Anhydrous) | | |
| L-Cysteine-HCl•H2O | 0.85 | Amino acid |
| Trace element | 10 mL | Trace metals |
| solution | | |
| PPG 2000 (2000 | 1 | Anti-foam |
| molecular weight | | |
| polypropylene glycol) | | |

TABLE 7
Fed Batch Phase Media

| Feed 1 | Conc (g/L) | Source |
|---|---|---|
| MgSO4 (g/L) | 1.07 | Provides ions helpful for growth of |
| | | bacteria |
| D(+) Glucose | 363.6 | Carbon |
| Feed 2 | Conc (g/L) | Source of |
| Hy soy | 150 | Nitrogen |
| Yeast BD | 30 | AA, carbon, nitrogen |
| Cysteine | 2 | AA |
| Base | Concentration(g/L) | Source |
| Na2CO3, 20% (w/v) | 200 | Base and carbon source |

### Example 2: Fermentation of S. pneumoniae in 90-95% N₂ and about 5-10% CO₂ Environments

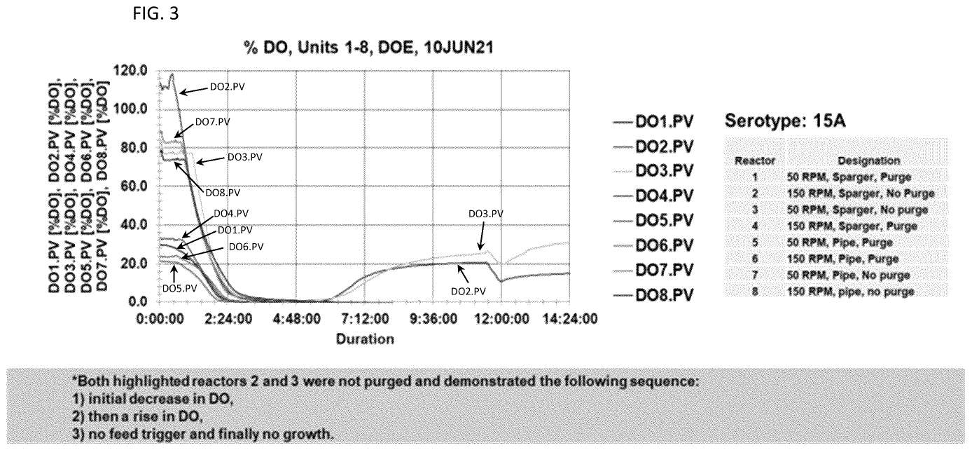

FIG. 3 shows the amount of dissolved oxygen (DO) for S. pneumoniae Serotype 15A under various experimental conditions. Of the eight runs, reactors 2 and 3 conditions led to cell death. This was likely because there was no purging prior to the run followed by the use of a sparger instead of a pipe assembly which gave an environment oxygen levels that exceeded the ideal level for cell growth. By comparison, the sparger provides tiny gaseous bubbles into the sample while the pipe assembly is able to deliver a large quantity of gas through the sample all at once. The benefits of using a pipe assembly as compared to using a sparger are especially noticeable when the reactor has not been purged (see curves for reactors 3 and 7). The curve for reactor 3 indicates that at 50 RPM, no purge and with sparging, the cells eventually died (starting at about 7 hours) compared to reactor 7, where also at 50 RPM, no purge but with the use of the pipe assembly, the cell continued to grow. The use of purging also assisted in promoting cell growth while the change in RPM values did not remedy the amount of DO in the reactors. For all of the instances where the cells in the reactor grew (reactors 1 and 4-8), the kLa was calculated to be about zero.

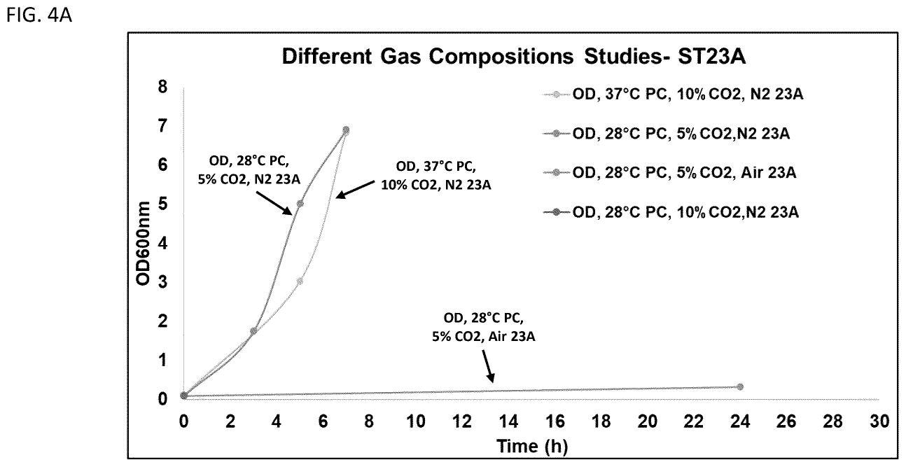

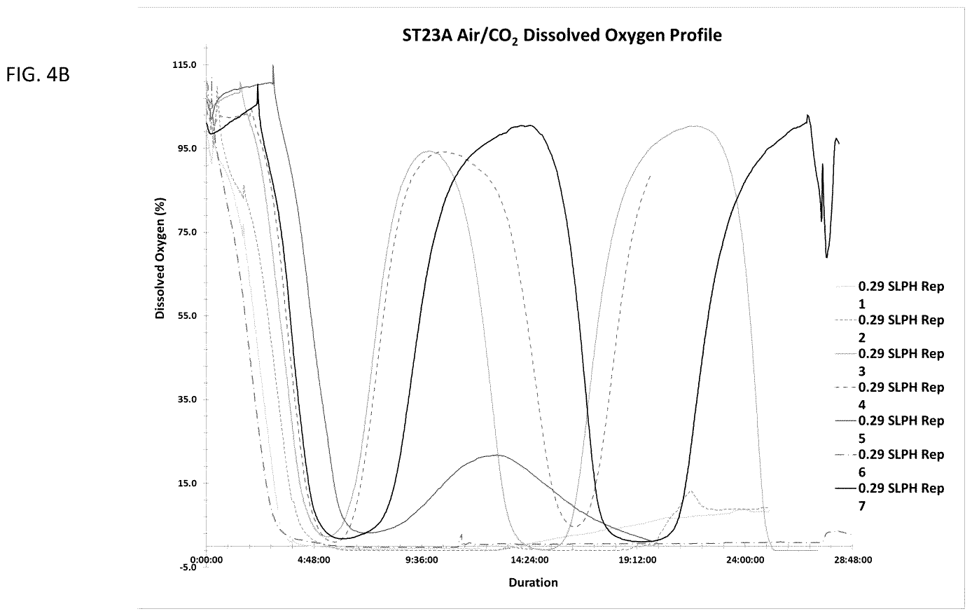

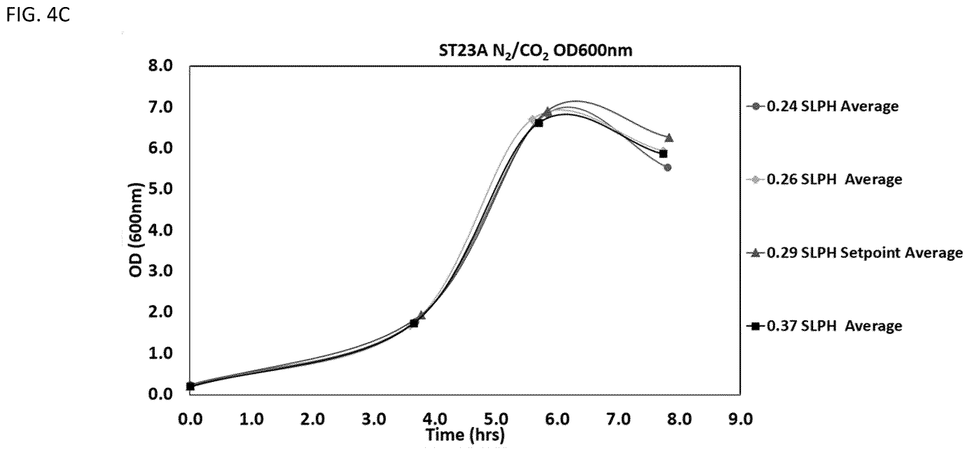

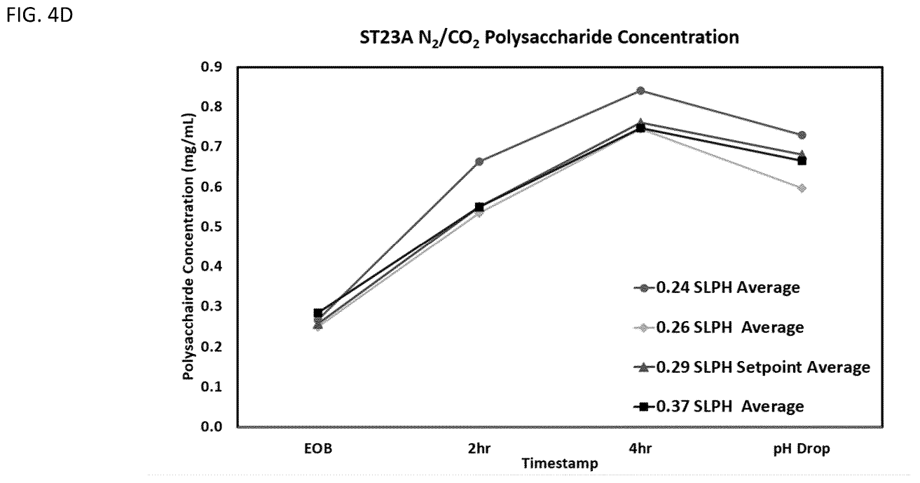

FIG. 4A shows growth curves for Streptococcus pneumoniae serotype 23A under conditions with and without oxygen and at two different temperatures. As the curves indicate, at temperatures of 28° C. and 37° C., Streptococcus pneumoniae serotype 23A had very similar growth profiles based on their respective OD_{600 nm} when the environment was either 5% CO₂:95% N₂ or 10% CO₂:90% N₂ (blue and yellow curves). Compared to the environment that had only CO₂ and N₂, the curve associated with 5% CO₂ and 95% air, showed that Streptococcus pneumoniae serotype 23A grew very minimally between the start and approximately 24 hours later.

FIG. 4B shows seven plots of dissolved oxygen over time from several fermentation runs in an air/CO₂ environment for Streptococcus pneumoniae serotype 23A at the same standard liter per hour (SLPH). From the seven repeat runs, it can be seen that even under the same conditions, there is no reproducibility with these runs. In some runs (Reps 1, 2, and 6), the fermentation was completed successfully, but in the remainder of the runs (Reps, 3, 4, 5, and 7), the fermentation runs did not complete successfully. The high variability of the results from fermentation runs under the 5% CO₂ and 95% ambient air conditions would make the manufacture of certain Streptococcus pneumoniae serotypes, like serotype 23 extremely unpredictable.

FIG. 4C shows plots of OD₆₀₀ over time from four fermentation runs for Streptococcus pneumoniae serotype 23A at four different flow rates of 95% N₂ and 5% CO₂. All four fermentation runs at the different gas flow rates show good repeatability in their growth profiles as compared to what was observed with the runs in the 5% CO₂ and 95% ambient air environment.

FIG. 4D shows plots of polysaccharide concentration over time for the four fermentation runs for Streptococcus pneumoniae serotype 23A at four different flow rates of 95% N₂ and 5% CO₂. Similar to the results when the OD₆₀₀ was measured over time, the polysaccharide plot profiles under the four different standard liters per hour (SLPH) were all fairly similar. The results from the fermentation runs under 95% N₂ and 5% CO₂ suggest that growth under these conditions provides for a predictable growth pattern for certain Streptococcus pneumoniae serotypes and production of these capsular polysaccharides.

Table 8 further shows the effect of growing conditions on serotype 23A. As the data indicate, when the environment in the reactor is free of oxygen, serotype 23A grows well while under air and CO₂ conditions, its growth is noticeably depressed at all variations of other parameters tested.

TABLE 8
Effect of various experimental conditions on serotype 23A growth.

| | | Gas Flow | | | kLₐ | OD₆₀₀ |
|---|---|---|---|---|---|---|
| | Gas | Rate | | | estimate | after |
| Unit | Mixture | (SLPH) | VVM | RPM | (/hr) | 72 hrs |
| 1 | 95% Air, | 0.5 | 0.05 | 300 | 3.8 | 0.29 |
| | 5% CO2 | | | | | |
| 3 | 95% N2, | 1.02 | 0.1 | 150 | 0 | 6.90 |
| | 5% CO2 | | | | | |
| 5 | 95% Air, | 0.51 | 0.05 | 150 | 2 | 0.25 |
| | 5% CO2 | | | | | |
| 6 | 95% Air, | 1.02 | 0.1 | 150 | 3.9 | 0.33 |
| | 5% CO2 | | | | | |
| 7 | 95% Air, | 2.04 | 0.2 | 150 | 5.9 | 0.38 |
| | 5% CO2 | | | | | |

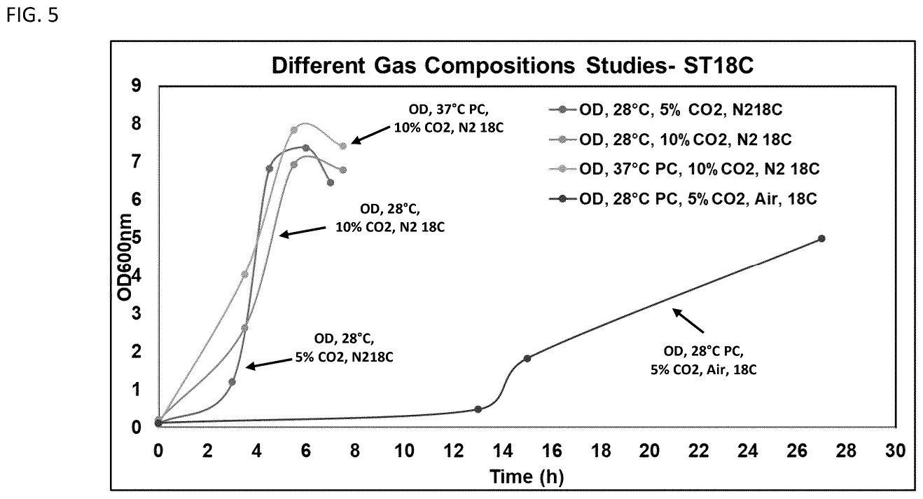

FIG. 5 shows similar growth profiles for Streptococcus pneumoniae serotype 18C. Similar to Streptococcus pneumoniae serotype 23A, Streptococcus pneumoniae serotype 18C grew well under varying conditions of temperature (28° C. and 37° C.) as well as varying conditions of gaseous environment (5% CO₂:95% N₂ and 10% CO₂:90% N₂). Also similar to Streptococcus pneumoniae serotype 23A, Streptococcus pneumoniae serotype 18C did not grow well in conditions where air was present (5% CO₂ and 95% air). Unlike Streptococcus pneumoniae serotype 23A, Streptococcus pneumoniae serotype 18C did show growth at approximately 13 hours after the initial start of fermentation that continued through to 28 hours after initiation of the fermentation process. Thus, it does appear that Streptococcus pneumoniae serotype 18C can grow in an environment with oxygen, it grows much faster in environments devoid of oxygen. Thus, for certain serotypes of aerotolerant bacteria, fermentation in oxygen-free environments provide improved growth as compared to environments where oxygen is present. This is critical in the manufacturing of bacterial vaccines (e.g., for S. pneumoniae) where a multitude of serotypes need to be prepared where serotypes that do not grow well in air would hold up production of the entire vaccine formulation.

1. A process for culturing aerotolerant bacteria for production of a biomolecule, the process comprising:
a) inoculating a culture medium with a colony or preculture of the aerotolerant bacteria in a bioreactor to form a bacterial culture;
b) supplying the bioreactor with a gaseous mixture substantially free of oxygen, wherein the gaseous mixture comprises at least one of nitrogen (N₂) and carbon dioxide (CO₂);
c) incubating the bacterial culture; and
d) maintaining a substantially oxygen-free environment in the bioreactor.

2. The process of claim 1, wherein the culture medium is an agar medium.

3. The process of claim 1 or 2, wherein there is no sugar, vegetable oil, hydrocarbon, alcohol, or organic acid added to the culture medium and/or bacterial culture.

4. The process of any one of claims 1-3, wherein the bacterial culture comprises at least one nitrogen source.

5. The process of any one of claims 1-4, wherein the bacterial culture comprises at least one nitrogen source selected from urea, ammonium hydroxide, ammonium salts, nitrates, amino acids, yeast extract, yeast autolysates, yeast nitrogen bases, protein hydrolysates, soybean meal, Hy-Soy, tryptic soy broth, cotton seed meal, malt extract, corn steep liquor, and molasses.

6. The process of any one of claims 1-5, wherein the bacterial culture comprises at least one phosphorus source.

7. The process of any one of claims 1-6, wherein the bacterial culture comprises at least one phosphorus source is a phosphate or polyphosphate.

8. The process of any one of claims 1-7, wherein the bacterial culture does not comprise an animal derived material.

9. The process of claim 8, wherein the animal-derived material is animal-derived heme or a prion protein.

10. The process of any one of claims 1-9, wherein the gaseous mixture substantially free of oxygen is supplied continuously until the biomolecule is formed.

11. The process of any one of claims 1-10, wherein the gaseous mixture substantially free of oxygen is supplied into a headspace of the bioreactor.

12. The process of any one of claims 1-10, wherein the gaseous mixture substantially free of oxygen is supplied directly into the culture medium and/or bacterial culture.

13. The process of any one of claims 1-12, further comprising the step of purging the bacterial culture with the gaseous mixture.

14. The process of claim 13, wherein the culture medium and/or bacterial culture is purged for at least 12 hours.

15. The process of any one of claims 13-14, wherein the bacterial culture is purged continuously until incubation is complete.

16. The process of any one of claims 1-15, wherein the gaseous mixture substantially free of oxygen comprises about 95% N₂ and about 5% CO₂.

17. The process of any one of claims 1-16, wherein the gaseous mixture substantially free of oxygen comprises about 90-95% N₂ and about 5-10% CO₂.

18. The process of any one of claims 1-17, wherein the gaseous mixture is substantially free of oxygen comprising a kLa value no more than 1.80.

19. The process of any one of claims 1-18, wherein incubating the bacterial culture comprises maintaining the temperature between about 30° C. and about 39° C.

20. The process of claim 19, wherein incubating the bacterial culture comprises maintaining the temperature at about 37° C.

21. The process of any one of claims 1-20, wherein incubation is complete when the bacterial culture reaches an OD₆₀₀ of at least 6.

22. The process of claims 1-21, wherein maintaining a substantially oxygen-free environment in the bioreactor comprises keeping oxygen concentration at 0 in the bacterial culture.

23. The process of any one of claims 1-22, wherein the aerotolerant bacteria are aerotolerant anaerobic bacteria.

24. The process of any one of claims 1-23, wherein the aerotolerant bacteria are S. pneumoniae or S. pyogenes.

25. The process of claim 24, wherein the aerotolerant bacteria are S. pneumoniae.

26. The process of claim 24 or 25, wherein the S. pneumoniae are of a serotype selected from 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20, 20A, 20B, 21, 22F, 23A, 23B, 23F, 24F, 31, 33F, 34, 35B, 35F, and 38.

27. The process of any one of claims 1-26, wherein the biomolecule is a capsular polysaccharide.

28. The process of claim 24, wherein the aerotolerant bacteria are S. pyogenes.

29. The process of claim 24 or 28, wherein the S. pyogenes is a serotype selected from M1, M2, M3, M4, M5, M6, M9, M11, M12, M13, M18, M22, M25, M28, M62, M71, M72, M74, M75, M77, M80, M81, M83, M87, M89, and M92.

30. The process of any one of claims 1-29, wherein the biomolecule is a cell wall polysaccharide.

31. The process of any one of claim 24 or 28-29, wherein the biomolecule is a polysaccharide or a variant thereof that lacks an immunodominant N-acetyl Glucosamine (GLcNAc) side chain.

32. The process of any one of claims 1-31, wherein the biomolecule is isolated.
